(19) United States
(12) Patent Application Publication
(10) Pub. No.: US 2023/0142314 A1
(43) Pub. Date: May 11, 2023

(54) Process Emulsification Simulator

(71) Applicant: Paul Robert Hart, League City, TX (US)

(72) Inventor: Paul Robert Hart, League City, TX (US)

(21) Appl. No.: 16/164,459

(22) Filed: Oct. 18, 2018

(51) Int. Cl. B01F 23/41; B01F 35/21; B01F 35/221; B01F 35/213

(52) U.S. Cl. CPC B01F 23/4105; B01F 35/2113; B01F 35/2215; B01F 35/213; B01F 2215/0409

(57) ABSTRACT

A small-scale, batch-wise device to simulate high-shear, short-duration emulsification of fluids from various industrial processes at elevated temperatures and pressures for the purpose of determining the quality and stability of those emulsions under different conditions and with different additives. A threaded, transparent tube capable high temperature and pressure is fitted with a threaded bearing with a shaft sealed gas tight at two points with spring-loaded, internally-facing, open rings. A socket head on the external end of the shaft held on a high-speed motor-drive rotates mixing blades on the internal end of the shaft. Process fluids and additives are added to the tube with a vaporizing liquid. The tube is sealed, heated to the process temperature under pressure, then inverted onto the motor drive, by which the blades are rotated at high-speed for a short duration. The tube is righted and the emulsion observed over time at process temperature under pressure.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO A SEQUENCE LISTING, A TABLE, OR A COMPUTER PROGRAM

Not applicable.

### BACKGROUND OF THE INVENTION

The present invention relates generally to the small-scale simulation of multiphase industrial processes, such as crude oil refinery desalters, oilfield free water knockouts and heater treaters, offshore 2-phase and 3-phase high and intermediate pressure separators, glycol and amine absorbers contaminated with hydrocarbon, or fuel lines contaminated with water; and more particularly, to the direct observation of the emulsification and demulsification of the included phases under the pressurized, high temperature, high shear, short duration conditions typical of the mix valves, control valves, and pressure chokes employed in these processes.

Many industrial processes involve making and breaking emulsions of oil and water. Crude, brine, and solids produced from geologic reservoirs form emulsions of both water-in-oil and oil-in-water as they pass through pumps, pressure chokes, and control valves. These emulsions must then be destabilized and separated in various surface vessels to obtain dry oil, clear water, and clean solids. The crude oil must also have residual salt and solids removed in the field or at refineries by adding wash water through a mix valve to make a fine emulsion of water-in-oil to extract the salt and wet the solids, then allowing that emulsion to break in a coalescer to remove the resultant brine with the solids. Produced or effluent water must have residual oil and solids removed, often by adding flotation gas through a dissolved gas choke or induced gas agitator to make fine bubbles of gas-in-water, which attach to the oil and solids; then allowing the laden bubbles to grow, rise, and break into a trough at the top, so that the resultant sludge or skim can be removed. Many other processes, though nominally a single phase of water or oil, become contaminated with an immiscible phase of oil or water, respectively, which then form troublesome emulsions as they pass through pumps, pressure chokes, and control valves.

Critical emulsion properties, such as particle size, are known to be independent functions of both the turbulence and the time expended in the formation of the emulsion, such that a low degree of turbulence for a long period of time is in no way equivalent, and indeed often has the opposite effect, as a high degree of turbulence for a short period of time. The turbulence is a known function of the external phase density and viscosity, which are known functions of temperature. Drop breakup and coalescence are known functions of the internal phase viscosity, the interfacial viscosity, and the interfacial tension, all of which are known functions of temperature and the types and amounts of surface-active additives present. Both the relative and absolute effect of these various additives, in turn, is a function of temperature, turbulence, and time. So, it is critically important, when simulating the creation and resolution of these process emulsions, that all three parameters—temperature, turbulence, and time—be simultaneously realistic, especially when testing chemical additives, which are also affected by these same parameters.

Additives may be added to improve or accelerate the aggregation or settling of the emulsified or dispersed phase to effect their ultimate separation. These additives, known as demulsifiers, emulsion breakers, reverse demulsifiers, reverse emulsion breakers, obverse demulsifiers, obverse emulsion breakers, solids setting aids, phase separation accelerators, defoamers, antifoams, dehydrators, deoilers, brighteners, clarifiers, coagulants, flocculants, coalescents, solids wetters, surfactants, or polymers are fed to one or the other phase, or both, to modify the oil/water/solids/gas interface. These additives increase the speed and/or completeness of the separation of oil, water, solids, and/or gas.

Additives may be added to impede or retard the aggregation or settling of the emulsified or dispersed phase to prevent their ultimate buildup. These additives, known as deposit inhibitors, dispersants, stabilizers, antifoulants, anti-settling aids, anti-agglomerants, emulsifiers, foamers, deliquifiers, or surfactants, are fed to one or the other phase, or both, to modify the oil/water/solids/gas interface. These additives decrease the speed and/or completeness of the separation of oil, water, solids, and/or gas.

Development of new chemical additives of these types has traditionally been done using a simple apparatus such as a glass bottle, jar, or tube that is shaken or stirred, then settled or centrifuged. These tests are referred to variously as bottle shaking, jar testing, settling or centrifuge testing, or by the tradename of the instrument used, such as TurbiScan® or LUMiSizer®. For electric field induced coalescence, a common test uses Petrolite's “Electric Desalter Demulsification Apparatus” (EDDA) available from InterAv, San Antonio, Tex. In this test, a batch of stable emulsion is made at room temperature by mixing 5 minutes or more in a high-speed blender. This is then poured into a set of conical tubes, each of which is then dosed with the additive intended to help make a fine but unstable emulsion (even though the emulsion is actually already made at that point.) They are only then heated in an aluminum block heater to a higher temperature, still below that of the process, to keep the water in the unsealed, unpressurized tubes from boiling. The water coalesces and settles in the presence of an internal electrode producing an unrealistic point source electric field. The rate of phase separation is monitored as a function of time by removing the tubes periodically from the block heater and observing or measuring by light transmission the amount of the settled phase that collects at the bottom of the vessel.

These methods have proven to be useful but they fail to adequately simulate the emulsification conditions of pressurized high temperature, high shear, and short duration, typical of the mix valves, control valves, and pressure chokes deployed in these processes. Using the wrong conditions creates the wrong emulsion which leads to the wrong conclusion being drawn. Failures are not uncommon.

US Patent Application 2012/0140213, 7-Jun-2012 describes a “Static Desalter Simulator”. It includes “an emulsion-forming device” and “a plurality of mixing tubes, each mixing tube having a cap member with a blending assembly configured to work with the emulsion-forming device to emulsify an oil/water mixture contained in the mixing tube”. The blending utilizes a realistic high shear of 10,000-16,000 rpm for a realistically short duration of 2-3 seconds.

The blending assembly consists of a “mixer-bushing”, a “central shaft”, and “mixer blades”. A bushing is just a protective liner, a thin tube or sleeve that allows relative motion by sliding. It is not a sealant or a seal. A CIP to the above application, 2012/0140058, 7-Jun-2012, adds to the description that “a sealing ring is positioned between the blade assembly and the measuring container to ensure a proper seal there between”. But no method is described for sealing the hole to the shaft while it is rotating at or even while it is still. Such a primitive bushing, as is commonly used on the commercial blenders described, cannot hold any pressure in the tube, and thus no temperature can be used above the boiling point of any of the fluid components, whether light hydrocarbons or water, without them actively boiling. This severely limits the utility of the described blending assembly.

Moreover, the mixer-bushing assembly is built into an internally-threaded cap for an externally-threaded tube, e.g. DIN GL-45. The cap is made of plastic: polyethylene, polyester, or thermoset resin, e.g. a “stirred reactor cap from Schott AG”. The problem with any plastic, or metal, wood, or even most ceramics is that they all have a higher coefficient of thermal expansion than borosilicate glass or quartz, the two transparent materials that could hold up to the temperature, pressures, and oil bath fluids to which the tube would be subjected. This means, when the capped tubes are heated, the caps expand more than the tubes, which loosens the caps. This causes them to leak when inverted to be mixed (the mixer is in the cap so it must be inverted), especially since inverting them causes a surge of pressure as condensate on the top is vaporized. But tightening the caps when they are hot and expanded causes them to shrink-wrap onto the threads when the tubes are cooled back down. We have discovered that the force of shrink wrapping is such that it makes it literally impossible to ever open them (the glass neck breaks first). If some extra measures, outside the patent, are taken to seal the gap in the bushing, so that it is under pressure when hot, we have discovered that loosening the cap when hot causes the water in the bottom to vaporize and push the oil out through the gap, spewing hot oil everywhere. Any method of sealing the tubes with such a cap makes using the tubes difficult and unsafe. The description neither recognizes nor solves this problem.

Another problem arises when heating the tubes from the bottom as with a bath (as called for in the method) or a hot plate. The top of the tube is then significantly colder than the fluid in the bottom. When a relatively non-volatile oil phase, such as a heavy crude oil, is added in conjunction with water, per the intended application to desalters, we have discovered that the water boils from the bottom and condenses in the top, refluxing back into the oil phase. The phase separation is churned and ruined. No method of overcoming this limitation is described. Not being able to use the method with such heavy crudes is a severe limitation.

An alternative, “Electrostatic Coalescer Testing Apparatus”, described in U.S. Pat. No. 5,529,675, 25-Jun-1996, Adamski, et al. overcomes some of these limitations at the expense of others. This apparatus uses a similar “mixer-bushing” in a plug held down by similar internally-threaded cap on an externally-threaded tube as the other does. It explicitly limits the inverted mixing to 80° C., since the bushing cannot hold pressure. The tube is placed inside a block heater, like an EDDA, but then a separate sealing plug is pushed into the bushing, and the heat-loosened cap pushed onto the tube with a cover plate over the block heater to seal the cap for the higher temperature, 120° C., settling part of the test. The covered block heater heats the top of the tubes as much as the bottom, so that vapor does not condense and cause refluxing. To observe the tubes, however, they must be cooled below boiling so the cover plate can be removed, the tubes lifted out of the block heater, observed, replaced, recovered, resealed, and reheated. But at the end of the test, at least the cold-hand-tight caps can be unscrewed.

Another issue is that the mixing blades extend the entire length of the tube, as they also serve as an internal electrode (like an EDDA). This requires a lower shear rate, under 10,000 rpm, produced with a plenary gear system. Combined with the higher viscosity at the lower blending temperature, emulsification with this device requires an unrealistic long duration of 2 minutes at the lower temperature and shear.

Another approach is the “Thermal Phase Separation Simulator”, U.S. Pat. No. 8,888,362, 18 Nov. 2014, Hart, et al. This uses a block heater, like the EDDA and Adamski '675, but with 12 wells for 12 bottles sealed with fluids from the start to take temperatures up to 150° C. at 110 psi. They are arranged in a circle with an observation window, so they do not have to be removed to be observed. Mixing is done by clamping the bottles into the block heater, turning it on its side, and shaking the entire assembly on a giant reciprocal shaker at speeds up to 240 rpm for a duration from 1 min to several hours. This does an excellent job of simulating pipe flow, the intended application. But it does not realistically simulate the fractional second to a few seconds of very high shear turbulence experience by fluid flow through a mix valve or pressure choke.

Larger, more elaborate, jacketed, instrumented, continuously stirred, gas pressurized, single cell devices are available, which are capable of mixing at the temperatures and pressures needed, for example, Series 5100 Glass Reactors from the Parr Instrument Company. These use sealed, jacketed reactors with magnetically coupled rotors. They are not sufficiently small or affordable for running many cells at once when continuous stirring is not required. Moreover, their maximum stirring speed is only 1700 rpm—not high enough to simulate the high shear, short duration process of flowing through a choke or valve.

One key parameter to simulate is the turbulent energy dissipation rate, ε,(m²/s³, J/kg-s, or W/kg), a measure of the kinetic energy of the turbulence, which dissipates progressively into ever smaller scale eddies. Droplets are most influenced by collisions with turbulent eddies of roughly the same size. Eddies can shear droplets apart or throw them together. Greater energy dissipation creates smaller eddies, which influence smaller droplets, which are the hardest to remove.

This turbulent energy dissipation rate, ε, can be calculated from the pressure drop, ΔP, and the time, t, over which the fluid experiences the pressure drop: ε=ΔP/ρt, where ρ is the fluid density. While the total energy dissipated is a measure of the amount of mixing, it is this dissipation rate that determines the nature of the mixing. The greater the pressure drop per unit time, the smaller the maximum droplet size that will be influenced. Thus, a pressure drop experienced over a short time, like through a choke or control valve, will influence a smaller droplet size than the same pressure drop experienced over a longer time, like in a pipeline, even if the total energy dissipated is the same.

For example, a 10-psi pressure drop over one second, typical of, say, a desalter mix valve, in a fluid with density and viscosity similar to water at the temperature of the process, produces an energy dissipation rate of about 70,000 J/kg-s and a total dissipated energy over that 1 second of about 70,000 J/kg.

Calculating ε for a cylindrical vessel stirred by a central rotating agitator is more complicated. It requires first calculating the Reynolds number Rₑ, to determine the turbulent flow regime, and from that the Fanning friction factor, ƒ. Rₑ=uD_{H}ρ/μ, where u=fluid velocity, D_{H} is the hydrodynamic diameter of the system, ρ is the fluid density, and μ is the fluid viscosity. Note that both density and viscosity are functions of the fluid temperature. For a cylindrical vessel stirred by a central rotating agitator, this translates to Rₑ=ND²ρ/μ, where u is the tip velocity of the agitator, D is the diameter of the agitator, and N is the rotational speed (s⁻¹). This system is fully turbulent for Rₑ>10,000. The Fanning friction factor can be estimated for different flow regimes in this case as follows:
For laminar flow (Rₑ<5700): ƒ=16/Rₑ
For transition flow (5200<Rₑ<10⁵): ƒ=0.0791/Re^{0.25}
For turbulent flow (10⁴<Rₑ<10⁷): ƒ=1.0014+0.125/Re^{0.32}
   The energy dissipation rate can then be calculated as ε=2ƒu³/D=2ƒN³D².

So, a 4-cm diameter blade rotating at 16,000 rpm, in a fluid with density and viscosity similar to water at the temperature of the mixing, produces an energy dissipation rate of about 70,000 J/kg-s. Blending for 1 second would dissipate a total energy of about 70,000 J/kg, same as the mix valve in the reference system. Since it is proportional to the cube of the rotational speed, mixing at 1700 rpm would be off by almost a factor of 1000.

To compare this with alternative mixing schemes, a mostly filled bottle in a reciprocal shaker uses a similar calculation: ε=2ƒu³/D=2ƒN³D², where u is the fluid velocity (ND), D is the throw of shaker plus the bottle head (up to length of throw, and N is the shaking speed (s⁻¹). For typical lab shakers with 1.5-inch throws, leaving 1.5 inches linear head in bottles of water-like fluid at room temperature, the maximum shaking speed of 280 rpm gives an ε of 1.2, off by a factor of about 60,000. If you shake it for 16 hours to dissipate the same total energy of about 70,000 J/kg, it will make 60,000 times too much of the wrong emulsion (the eddy size produced depending only on the dissipation rate).

Thus, it can be seen from these fluid mechanical requirements that it is simply is not possible to simulate the emulsification at a choke point or valve with anything less than the right temperature (ρ, μ), turbulence (Rₑ) and time (ε). And getting the right temperature requires the mixing vessel to be sealed and pressurized when it is being mixed at high shear with a high-speed internal agitator.

A method of doing so, in order to improve the methods of simulating process emulsification such that one may better select the additive chemistries and/or operating parameters needed to optimize the process, will now be described.

### BRIEF SUMMARY OF THE INVENTION

In one aspect, the invention is directed to a small-scale, batch-wise device to realistically simulate the high-shear, short-duration emulsification of fluids occurring in various industrial processes at elevated temperatures and pressures for the purpose of seeing the quality and stability of those emulsions under different conditions and with different additives. An internally-threaded, transparent tube capable of withstanding temperatures up to 220° C. and pressures up to 200 psi is fitted with a threaded bearing with a shaft sealed gas tight at both the top and bottom with spring-loaded, facing, open rings. Mixing blades are attached to the internal end of the shaft and a socket head connected to the external end of the shaft. In one embodiment, the tube is internally-threaded and the bearing is a plug that is externally-threaded with a coefficient of thermal expansion greater than that of the tube, allowing the closure to tighten when heated, to hold pressure, and loosen when cooled, to easily open. In one embodiment, the dual seals are at least 1 cm apart to allow the inserted rotatable shaft to remain sealed when manually engaged.

In another aspect, the invention is directed to a method of using this device to realistically simulate the emulsification and demulsification of immiscible phases under the simultaneous conditions of high temperature, high pressure, high shear, and short duration typical of industrial processes employing mix valves, control valves, and pressure chokes. The process fluids are added to the tube. The tube is sealed with the bearing, heated to the temperature of the process and enough pressure to prevent boiling, then inverted and the socket placed on a motor drive, which is then rotated at high-speed for a short duration. The tube is righted and the emulsion observed over time at the temperature of the process and a pressure adequate to prevent boiling. In one embodiment, the tube is heated from the bottom, with a hot plate or in a bath of a thermal transfer medium, and an inert, vaporizing liquid is added to the tubes to maintain enough pressure to suppress refluxing of low boiling process fluids from the cooler top of the tubes.

The present invention and its advantages over the prior art will become apparent upon reading the following detailed description and the appended claims with reference to the accompanying drawings.

### BRIEF DESCRIPTION OF THE DRAWINGS

FIG. 1 illustrates a process emulsification simulator apparatus comprising immiscible fluids inside an inverted mixing tube with a sealed blending mechanism attached to a speed- and time-controlled motor drive.

FIG. 2 illustrates a mixing tube comprised of three parts, an internally-threaded, circular cross-section top, a non-circular cross-section middle (two examples shown), and a closed bottom of any cross-section and taper (three examples shown).

FIG. 3 illustrates a mixing plug assembly that screws into the mixing tube of FIG. 1, comprised of an externally-threaded bearing with two spring loaded seals holding a concentric, bladed shaft.

FIG. 4 illustrates a close-up cutaway of a spring-loaded, open-face rotary seal.

FIG. 5 illustrates a blender base comprising a motor drive with controls for adjusting the speed and timing of its rotation.

Corresponding reference numbers indicate corresponding parts throughout the views of the drawings.

### DETAILED DESCRIPTION OF THE INVENTION

The invention will now be described in the following detailed description with reference to the drawings, wherein preferred embodiments are described in detail to enable practice of the invention. Although the invention is described with reference to these specific preferred embodiments, it will be understood that the invention is not limited to these preferred embodiments. But to the contrary, the invention includes numerous alternatives, modifications, and equivalents as will become apparent from consideration of the following detailed description.

A process emulsification simulator apparatus, 10, provides the ability to test chemical additives on process fluids using realistic temperatures, pressures, shear, and duration of turbulent process flow. The process emulsification simulator apparatus, 10, uses small amounts of process fluids to perform the experiments, thereby reducing the cost of sampling, transport, and disposal. In the process emulsification simulator apparatus, 10, process fluids are added to the mixing tube, 20, chemical additives are added to the process fluids, the tube is sealed with the mixing plug, 30, the tube and contents are heated to the process temperature and vapor pressure by conventional means, the tube is inverted onto the blender base, 50, and the fluids are mixed together at this temperature with a shear and duration equivalent to that of the valve or choke in the industrial process being simulated. Then the tube is righted, returned to the conventional means of heating to the process temperature, and the emulsion so formed is allowed to coalesce and settle in the same tube at the temperature, vapor pressure, electric field strength and geometry that may apply, for a residence time equivalent to that part of the process. Conventional means of heating include immersion in a bath of oil, glycol, sand or other media, contact with a block heater or hot plate, radiant or convective transfer in a thermal or microwave oven, or any other convenient means. Likewise, settling after mixing may be in a bath of oil, glycol, sand or other media, in a block heater, on a hot plate, in a thermal or microwave oven, or any other convenient device. Application of a coalescent electric field may be done by immersion of the tube in an external electric field of the appropriate strength, frequency, and geometry, produced, for example, by parallel-plate electrodes placed outside the tubes.

### EXAMPLE

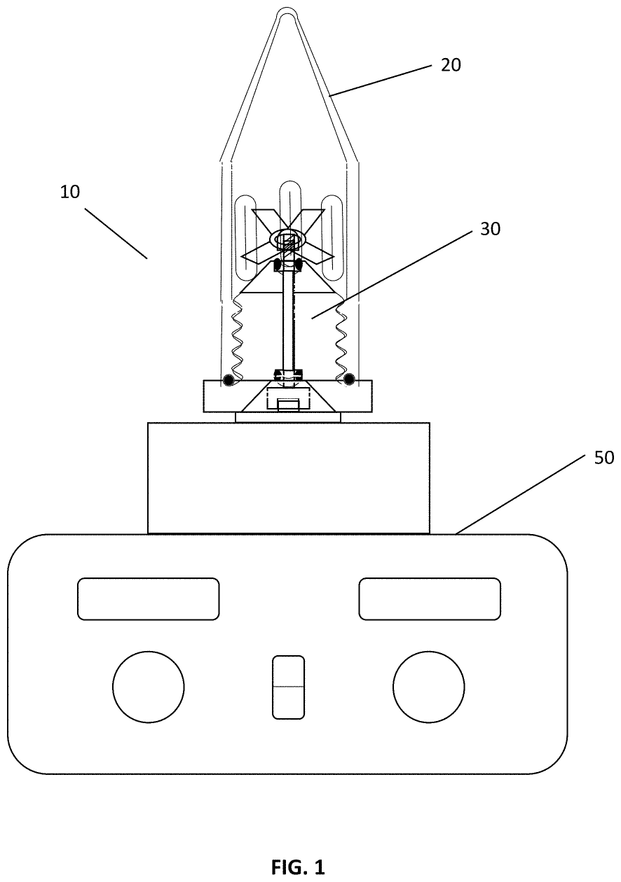

Referring now to FIG. 1, the process emulsification simulator apparatus, 10, contains a mixing tube, 20, made of borosilicate glass, quartz, sapphire, or other substantially transparent material that can withstand process temperatures and pressures up to at least 150° C. and 100 psi, preferably 220° C. and 200 psi, in air or immersed in an appropriate thermal transfer fluid, like silicone oil. The mixing tube, 12, is desirably made of a transparent material so that the operator may visually monitor the state of the emulsion in the tubes to obtain experimental results. However, other means of monitoring through non-visually transparent materials with internal or external sensors are also possible.

The mixing tube, 20, is closed with a mixing plug assembly, 30, made of a process-fluid compatible, high-temperature capable, machinable or moldable material with a coefficient of thermal expansion greater than that of the tube, such as metal or plastic. Process-fluid compatible and high-temperature capable means that it does not dissolve, soften, or degrade on contact with process fluids, like oil and water, at process temperatures above 100° C. Examples of such plastics include various polyesters, polyamides, polyacetals, poly(melamine-formaldehyde), polytetrafluoroethylene (PTFE), polyphenylene sulfide (PPS), and polyetheretherketone (PEEK). Examples of appropriate metals include brass and stainless steel. Plastic is preferred to metal, as it conducts less heat and has lower heat capacity, making it cooler to the touch and thus easier to handle by hand. Plastic also has lower surface hardness and will not scratch the glass, which weakens it under pressure. The preferred plastic is reinforced or filled with particles or fibers for dimensional stability and lubricity. An example is VERTEC® 5025, and internally lubricated, carbon fiber reinforced PEEK.

Having a coefficient of thermal expansion greater than that of the tube ensures that as the tube is heated, the plug expands more, which tightens the seal, and as it cools back down, the plug shrinks back, allowing the tube to be easily opened. Representative coefficients of thermal expansion, in 10⁻⁶/° F., of tube materials include quartz at less than 1 and borosilicate glass at about 4. Representative coefficients of thermal expansion, in 10-6/° F., of plug materials include Stainless Steel at about 16, PEEK at about 25, and PTFE at about 112.

The mixing plug, 30, is in contact with a blender base, 50, which supplies the motive force for mixing the fluids in the mixing tube, 20. In one embodiment, the blender base, 50, is a variable speed, timing selectable commercial blender, capable of speeds of at least 10,000 rpm, for example a Waring®, Vitamix®, Blendtec®, or Kitchen Aid® blender. In one embodiment, the blender base has pre-set, push button speed settings in the range of 3000 to 24,000 rpm. In one embodiment, the mixing speed is controlled by a variable transformer connected to the blender motor. In one embodiment, the duration of mixing is controlled by any conventional electronic timer suitable for precision timing of the on/off switching of an electrical appliance. Suitable external timers are available from GraLab® of Centerville, Ohio. In this way, the operator can select the speed and duration of the rotation to vary the tightness of the emulsion, to match that of the process being simulated.

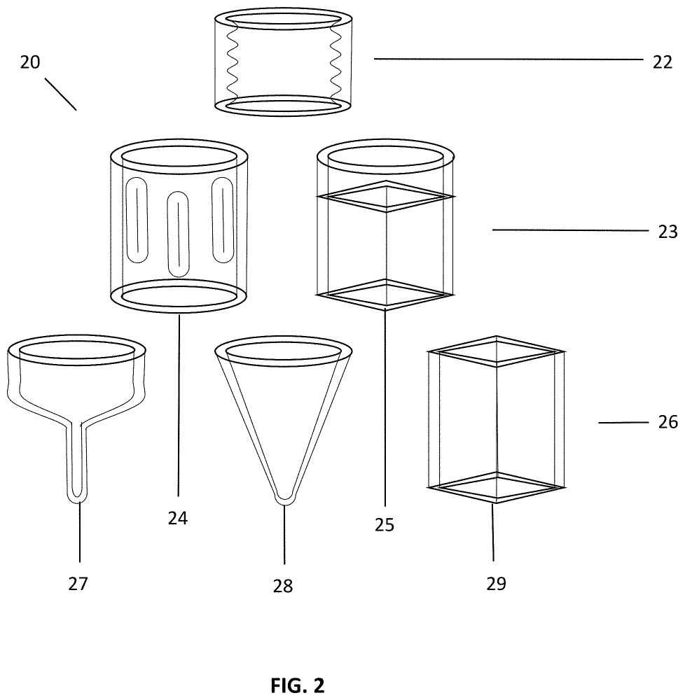

Referring to FIG. 2, the mixing tube, 20, is desirably made of borosilicate glass, e.g. Pyrex® or Duran®, since this is easy to form, permits visible inspection, and prevents any significant electrical conduction if immersed in an electric field. The mixing tube, 20, is of sufficient thickness to not break under normal usage at the temperature and pressures applied in apparatus, 10. A two-inch (50.8 mm) OD tube requires a “Medium” wall thickness of 0.126 inch (3.2 mm) to hold 105 psi, and a “Heavy” wall thickness of 0.228 inch (5.8 mm) to withstand 230 psi. The volume of the tube can vary from 50 to 250 mL. The tube has a maximum fill line at 80-90% of its capacity, allowing 10-20% head space for thermal expansion of the liquid. About 100 mL to the fill line with another 20 mL head space when sealed is a convenient size. The tube is desirably graduated below the fill line to facilitate direct observation of the volume of the fluids contained therein.

The shape of the tubes can be divided into three sections, each serving a different purpose. The top section, 22, features a circular cross section and internal threads to fit the plug, 30. The middle section, 23, where the fluid mixes, has a non-circular cross section to prevent vortexing of the fluid when mixed. Vortexing produces an unrealistic, emulsion breaking, centrifugal force. In one embodiment, the non-circular cross section comprises trigonal Morton indentations or vanes, 24. Single or double indentations are also possible. In one embodiment, the non-circular cross section comprises a tetragonal or square cross section below the circular connection to the top section, 25. In one embodiment, the non-circular cross section comprises a pentagonal cross section below the circular connection to the top section. In one embodiment, the non-circular cross section comprises a hexagonal cross section below the circular connection to the top section. Any substantially non-circular cross section will do. The bottom section, 26, where the fluid separation is mostly observed and measured, desirably has a cross section to match up with the middle section, 23. But a transition to a different cross section is also possible. In this section, the taper of the tube to closure can be varied to optimize the accuracy of reading the amount of the heavier phase that has fallen to the bottom, in an oil-water system, typically called the “water drop”. Smaller water drops are more accurately measured with a narrow diameter “receptacle tip”, 27. Medium water drops are more accurately measured with a conical bottom, 28. Larger water drops, or smaller oil rises, are more accurately measured with a flat bottom, 28. A round bottom is also a possibility. The top section, 22, can be attached via glass fusion to any of the middle sections, 23, which can be attached by glass fusion to any of the bottom sections, 26.

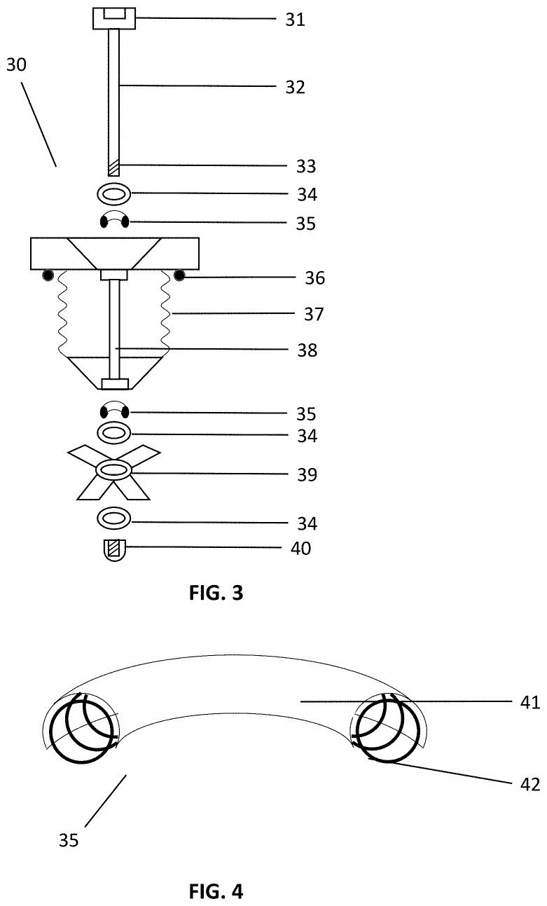

FIG. 3 shows in detail the mixing plug assembly, 30. A base plug, 37, threads into the mixing tube, 20, and seals the open end of the mixing tube, 20, to the bottom lip of the base plug, 37, with an O-ring, 36. The O-ring can be made of Viton®, an inert, high temperature fluoroelastomer, or any process-fluid compatible, high-temperature capable polymer or elastomer. The base plug, 37, of the plug assembly, 30, is bored with a hole, 38, for a shaft, 32, to be inserted through it. The shaft, 32, is preferably made of a hard metal, e.g. stainless steel, the surface of which has desirably been chromed or otherwise further hardened and polished to a high degree of smoothness, e.g. a mirror finish. The shaft, 32, has a socket head, 31, on the external end and threads, 33, on the internal end. The shaft, 32, is inserted through the hole, 38, in the plug, 37, and held in place at both the external and internal sides of the plug, 37, with two spring-loaded, open-faced rotary seals, 35, facing the internal (high pressure) side of the plug, 37. Washers, 34, are placed above the external seal, 35, and below the internal seal, 35. These washers are preferably made of an inert, low friction material, such as PTFE, or a hard metal, such as stainless steel. A mixing blade, 39, is held to the lip of the threads, 33, in the shaft, 32, with a washer, 34, and an acorn nut, 39. The mixing blade, 39, and acorn nut, 40, are preferably made of the same or similar hard metal as the shaft, 32. The mixing blade, 39, can be any type of foil, paddle, vane, propeller, impeller, or rotor/stator as needed to simulate the turbulence of a given process. Typically, a 4-fin stainless steel foil blade, available from Waring, may be used.

Referring now to FIG. 4, the rotary seals, 35, are comprised of an outer, open-face sheath, 41, and an inner, spring coil, 42. The sheath, 41, is desirably made of a low friction material, such as a fluoropolymer, like PTFE, preferably reinforced with glass, graphite, or carbon fiber for wear resistance. The spring, 42, is desirably made with a fluid-compatible, high strength material, such as stainless steel. Both sheaths, 41, open toward the internal, higher pressure side of the plug, so that increasing pressure increases the tightness of the seal. Rotary seals of this type are available from Ball Seal Engineering, Inc., Foothill Ranch, Calif.

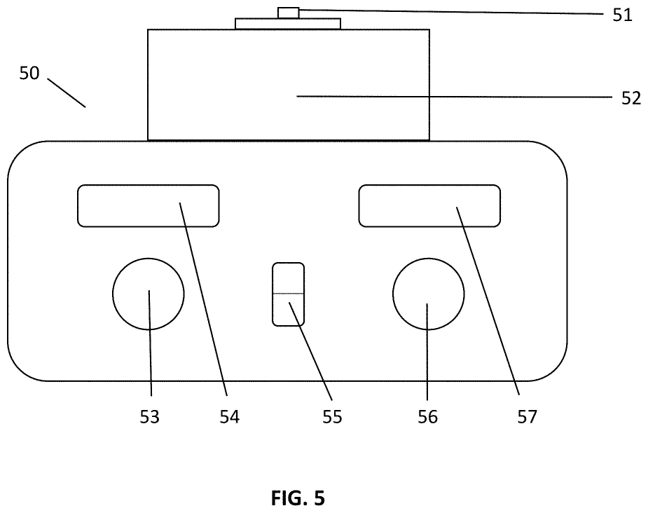

Although one seal of this type is capable of sealing a high speed rotating shaft when the shaft is orthogonally aligned with the seal, manually holding the socket head, 31, onto the drive head, 51, shown in FIG. 5, does not align the shaft, 32, orthogonally with the hole, 38, and thus the seal, 35. In order to do so, a second seal, spaced a certain minimum distance apart, was found to be necessary to make the shaft, 32, gas tight, while being held on the drive head, 51, manually and rotated at high speed. Even slightly off-center positioning or sideways movement of the socket head, 31, during this manual operation caused enough distortion to a single seal, or two seals placed too close together, to lose the vapor tightness during high speed mixing, as well as damage the seal itself. Even a small loss of pressure can cause catastrophic vaporization and liquid ejection from the tube. The minimum spacing, center to center, was found to be about 0.5 cm. Preferably spacing is at least 1.0 cm.

Referring now to FIG. 5, the blender base, 50, comprises a motor drive, 52, for turning the drive head, 51, controlled by a speed controller, 53, which could be a knob, as shown, or a series of discrete speed push buttons, and a timing controller, 56. In one embodiment, the speed and timing controls are built into the blender base, 50, as one device. In one embodiment, the speed controller, 53, and/or the timing controller, 56, are separate devices connected to the blender base, 50. A suitable separate speed controller 53 might be a variable voltage transformer, e.g. a Variac®. A suitable separate timing controller 56 might be a Model 451 Intervalometer from GraLab® of Centerville, Ohio. The speed controller can vary the rotational speed from 3000 to 30,000 rpm. The timing controller can control the duration of the rotation from 0.1 seconds to 100 seconds. The speed controller may optionally include a speed setting display, 54. The timing controller may optionally in include a timer setting display, 57. Once the speed and timing are set, an on/off switch, 55, initiates the blending. The on/off switch might be integrated with the motor drive, 52, the speed controller 54, or the timing controller 57. Suitable blender bases are available from Waring®, Vitamix®, Blendtec®, and Kitchen Aid® among others.

The invention is also directed to a method of simulating process emulsification to select phase separation control additives for multiphase industrial process. In one embodiment, the same phase ratio as found in the multiphase system to be modeled is added, and the same temperature, turbulent energy dissipation rate, and duration of turbulence as found in the process is used to make the emulsion or dispersion and allow it to separate. Then the amount of each phase that separates from the emulsion or dispersion so formed as a function of time is measured. The additive with the slowest and least separation is selected as the best emulsifier, dispersant, antifoulant, or deposit inhibitor; the additive with the fastest and most separation is selected as the best demulsifier, coalescent, coagulant, or flocculent.

In performing such tests where temperature is maintained by heating from the bottom of the tube, one embodiment of the method is the addition to the tube before it is sealed of an inert vaporizing liquid, lower boiling than the lowest boiling process phase, to prevent top-tube condensation and refluxing of the lowest boiling process phase. Inert means it does not interact with any process fluid to the extent that that would significantly inhibit its ability to vaporize at the process temperature. Suitable inert vaporizing liquids include fluoro-, chloro-, and hydro-carbons, ethers and esters. In oil-water systems, the preferred vaporizing liquids are pentane, hexane, and heptane.

### EXAMPLE

In order to assess the salt extraction efficacy of candidate extraction aids, simulated refinery desalter tests were undertaken using the process emulsification simulator apparatus, 10.

The conditions of the process:
1. Process temperature: 140° C.
2. Wash water ratio: 5% of total charge
3. Mix valve differential pressure: 10 psi
4. Mix valve transit time: 1 second
5. Electric field strength, frequency, and orientation: 4 kV/inch, AC 60 Hz, vertical
6. Oil phase residence time: 16 minutes

Preliminary measurements:
1. Vapor pressure of crude oil at process temp: 12 psi
2. Vapor pressure of wash water at process temp: 38 psi
3. Vapor pressure of 5% hexane in crude oil at process temp: 55 psi

Procedure:
1. Pre-heat a silicone bath with tube fitting, horizontal electrode rack to 150° C.
2. Add 5 mL wash water to the mixing tube, 20.
3. Add reverse emulsion breaker candidate to the water.
4. Add 95 mL crude oil to the tube.
5. Add salt extraction aid candidate to the oil.
6. Add 5 mL hexane to the oil.
7. Seal the mixing tube, 20, with the mixing plug assembly, 30.
8. Place sealed mixing tube in rack in bath.
9. Set blender speed, 53, to 16,000 rpm and timer duration, 56, for 1 second.
10. Set electrical field on horizontal plates of electrode rack to 4 kV/inch, AC 60 Hz.
11. After 15 minutes in bath, turn bath temperature down to 140° C.
12. Pick up tube by the plug, 37, using oil-proof, thermal gloves, and invert on blender base, 50.
13. Turn on blender switch, 55, to initiate pre-set timer and speed.
14. Replace mix tube, 20, in rack in bath.
15. Measure the amount and quality of separated water and interface with the oil.
16. Repeat same measurements after 1, 2, 4, 8, 16, and 32 minutes (twice the residence time).

The best candidate for this application has no water initially, followed by the most rapid and complete separation of all 5 mL of water, with no rag layer and no oil emulsified in the water.

Accordingly, the process emulsion simulator apparatus, 10, permits the operator to simulate useful parameters including but not limited to: process temperature, fluid densities and viscosities, vapor pressure, and rate and duration of turbulent energy dispersion. The emulsion is formed in the presence of additives at temperature and pressure in a mixing tube, 20, sealed with a mixing plug assembly, 30, which tightens when it is heated and loosens when it cools. The emulsion is resolved at temperature and pressure in the same tube without disruptive refluxing of lower boiling phases. The quantity and quality of the emulsification and demulsification can be visually verified and measured as a function of time to select appropriate additives or conditions for the process.

While the disclosure has been illustrated and described in typical embodiments, it is not intended to be limited to the details shown, since various modifications and substitutions can be made without departing in any way from the spirit of the present disclosure. As such, further modifications and equivalents of the disclosure herein disclosed may occur to persons skilled in the art using no more than routine experimentation, and all such modifications, equivalents, and any and all possible combinations of some or all of the various embodiments are believed to be within the scope of the disclosure as defined by the following claims.

All of the compositions and methods disclosed and claimed herein can be made and executed without undue experimentation in light of the present disclosure. While this invention may be embodied in many different forms, there are described in detail herein specific preferred embodiments of the invention. In addition, unless expressly stated to the contrary, use of the term “a” is intended to include “at least one” or “one or more.” For example, “a device” is intended to include “at least one device” or “one or more devices.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

1. An apparatus for testing emulsification of immiscible phases in a process, comprising:
a threaded, transparent tube fitted with a threaded bearing, said bearing being sealed gas tight to the tube with an O-ring and sealed gas tight at both the top and bottom of an inserted shaft with dual, spring-loaded, internally-facing, open rings by which said shaft remains sealed when manually engaged and rotated at high-speed;
said shaft having mixing blades attached to its internal end and a socket head connected to its external end;
said the socket head fitting a drive head on a speed- and time-controlled rotary motor drive.

2. An apparatus of claim 1 in which
the threaded transparent tube is internally-threaded and
the threaded bearing is an externally-threaded plug.

3. An apparatus of claim 2 in which the material of the plug has a higher coefficient of thermal expansion than the material of the tube.

4. An apparatus of claim 3 in which
the material of the plug is comprised of metal or plastic and
the material of the tube is comprised of ceramic or glass.

5. An apparatus of claim 4 in which
the material of the plug is comprised of process-fluid compatible, high-temperature capable plastic and
the material of the tube is comprised of borosilicate glass.

6. An apparatus of claim 1 in which the dual, spring-loaded, internally-facing, open rings are spaced at least 0.5 cm apart.

7. An apparatus of claim 1 in which the sheath material of the spring-loaded, internally-facing, open rings is comprised of reinforced fluoropolymer.

8. An apparatus of claim 1 in which at least the middle section of the transparent tube has a non-circular cross section.

9. A method of simulating emulsification of immiscible phases in a process, comprising:
determining the vapor pressure at process temperature of each immiscible phase;
adding the phases to a threaded, transparent tube along with an inert liquid with a lower boiling point than the lowest boiling phase;
sealing the fluids in the tube with a threaded bearing sealed to the tube with an O-ring with a inserted, rotary shaft held and sealed gas tight at both the external and internal side of the bearing with dual, spring-loaded, internally-facing, open rings, where mixing blades are attached to the internal end of the shaft and a socket head connected to the external end of the shaft;
heating the fluids in the tube to the process temperature and an adequate vapor pressure to prevent boiling;
manually inverting the tube and engaging the socket in the drive head of a speed-and time-controlled rotary motor drive;
rotating with a shear and for a duration approximating that of the emulsifying element of the process;
righting the tube, and
measuring the degree of emulsification and its subsequent rate of resolution at the process temperature and adequate vapor pressure to prevent boiling of the process fluids.

10. A method of claim 9 in which
the threaded transparent tube is internally-threaded and
the threaded bearing is an externally-threaded plug.

11. A method of claim 10 in which the material of the plug has a higher coefficient of thermal expansion than the material of the tube.

12. A method of claim 11 in which
the material of the plug is comprised of metal or plastic and
the material of the tube is comprised of ceramic or glass.

13. A method of claim 12 in which
the material of the plug is comprised of process-fluid compatible, high-temperature capable plastic and
the material of the tube is comprised of borosilicate glass.

14. A method of claim 9 in which the dual, spring-loaded, internally-facing, open rings are spaced at least 0.5 cm apart.

15. A method of claim 9 in which the material of the spring-loaded, internally-facing, open rings is comprised of a reinforced fluoropolymer.

16. A method of claim 9 in which at least the middle section of the transparent tube has a non-circular cross section.

17. A method of claim 9 in which the inert liquid is a fluorinated, chlorinated, and/or hydrogenated carbon, ether, or ester.

18. A method of claim 17 in which the inert liquid is pentane, hexane, or heptane.
